SEQUENCE LISTING (8 sequences)

SEQ ID NO: 1 - DNA, 723 bp, Artificial Sequence
  EC-SOD sequence
atgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcctga

SEQ ID NO: 2 - protein, 240 aa, Artificial Sequence
  EC-SOD protein
MLALLCSCLLLAAGASDAWTGEDSAEPNSDSAEWIRDMYAKVTEIWQEVMQRRDDDGTLHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSERKKRRRESECKAA

SEQ ID NO: 3 - DNA, 681 bp, Artificial Sequence
  209E mutant sequence
atgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagag

SEQ ID NO: 4 - protein, 227 aa, Artificial Sequence
  209E mutant protein
MLALLCSCLLLAAGASDAWTGEDSAEPNSDSAEWIRDMYAKVTEIWQEVMQRRDDDGTLHAACQVQPSATLDAAQPRVTGVVLFRQLAPRAKLDAFFALEGFPTEPNSSSRAIHVHQFGDLSQGCESTGPHYNPLAVPHPQHPGDFGNFAVRDGSLWRYRAGLAASLAGPHSIVGRAVVVHAGEDDLGRGGNQASVENGNAGRRLACCVVGVCGPGLWERQAREHSE

SEQ ID NO: 5 - DNA, 1106 bp, Artificial Sequence
  SOD3 - GFP sequence
agcttgatttccgggggcctctagactacttgtatagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatctcgagtcctccggcggccttgcactcgctctcgcgccgccgcttcttgcgctctgagtgctcccgcgcctggcgctcccagagcccgggcccgcacacgcccaccacgcagcaggccagccgccggcccgcgttcccgttctccacgctggcctggttgccgccgcggcccaggtcgtcctcgcagcgtggacgaccacggcccggcccacgatggagtgcgggccgcgagcgagcgccaggccggcgcggtacctccagaggctgcgtcgcggacgcaagttgccgagtcgcccgggtgctgcggggtgcgcacggccagcggttgtagtgggcccggtgaactcgcagccctggttag

SEQ ID NO: 6 - DNA, 753 bp, Artificial Sequence
  SOD3 - His6tag sequence
aagcttatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcgcaagaagcggcggcgcgagagcgagtgcaaggccgcccatcatcatcatcatcattaatctaga

SEQ ID NO: 7 - DNA, 1198 bp, Artificial Sequence
  209E-GFP sequence
ccagaacatggcaagcgtttaacttaagcttggtaccgagctcggatccactagtccagtgtggtggaattcgccatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcgcgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagggaggactcgagatggtgagcaagggcgaggagctgttcaccgggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgagcagcacgacttcttcaggtccgccatgcccggaggctacgtccagagcgccccatctttcttcagacgacgcactacagacccgcgcgccgaaggggaagttcgagggcgaacccctgtgaaccgcatcgagctgatggcatcgacttcaggaggacgcaccatcctggggcacagcctggattcacttt

SEQ ID NO: 8 - DNA, 714 bp, Artificial Sequence
  209E-His6Tag sequence
aagcttatgctggcgctactgtgttcctgcctgctcctggcagccggtgcctcggacgcctggacgggcgaggactcggcggagcccaactctgactcggcggagtggatccgagacatgtacgccaaggtcacggagatctggcaggaggtcatgcagcggcgggacgacgacggcacgctccacgccgcctgccaggtgcagccgtcggccacgctggacgccgcgcagccccgggtgaccggcgtcgtcctcttccggcagcttgcgccccgcgccaagctcgacgccttcttcgccctggagggcttcccgaccgagccgaacagctccagccgcgccatccacgtgcaccagttcggggacctgagccagggctgcgagtccaccgggccccactacaacccgctggccgtgccgcacccgcagcacccgggcgacttcggcaacttcgcggtccgcgacggcagcctctggaggtaccgcgccggcctggccgcctcgctcgcgggcccgcactccatcgtgggccgggccgtggtcgtccacgctggcgaggacgacctgggccgcggcggcaaccaggccagcgtggagaacgggaacgcgggccggcggctggcctgctgcgtggtgggcgtgtgcgggcccgggctctgggagcgccaggcgcgggagcactcagagcatcatcatcatcatcattaatctaga

(19) United States
(12) Patent Application Publication
(10) Pub. No.: US 2016/0108390 A1
(43) Pub. Date: Apr. 21, 2016

(54) COMPOSITION FOR STABILIZING EC-SOD AND METHOD OF STABILIZING EC-SOD USING THE SAME

(71) Applicant: The CATHOLIC UNIVERSITY OF KOREA INDUSTRY-ACADEMIC COOPERATION FOUNDATION, Seoul (KR)

(72) Inventors: Tae-Yoon KIM, Seoul (KR); Sunghwan Kim, Seoul (KR); Hae-Young Kim, Seoul (KR); Jung-Ho Kim, Seoul (KR); Jung-Hye Choi, Seoul (KR); Won-Kook Ham, Seoul (KR); Yoon-Jae Jeon, Seoul (KR)

(21) Appl. No.: 14/721,759

(22) Filed: May 26, 2015

(30) Foreign Application Priority Data
Oct. 21, 2014 (KR) 10-2014-0142624

(51) Int. Cl. C12N 9/96; C12N 9/02

(52) U.S. Cl. CPC C12N 9/96; C12N 9/0089; C12Y 115/01001

(57) ABSTRACT

The present invention relates to a composition for stabilizing extracellular superoxide dismutase (EC-SOD) protein and a method for stabilizing EC-SOD protein using the composition. More particularly, the present invention relates to a composition for stabilizing EC-SOD protein comprising bovine serum albumin (BSA), human serum albumin (HSA), polyethylene glycol (PEG) or fetal bovine serum (FBS) as an effective ingredient; a method for stabilizing EC-SOD protein using the inventive composition; and a method for preparing a stabilized EC-SOD protein using the inventive composition.

The present invention provides a composition for stabilizing EC-SOD protein and a method for stabilizing EC-SOD using the inventive composition in order to use EC-SOD protein for clinical or non-clinical purposes. Further, the present invention attributes to the obtainment of stabilized EC-SOD protein, leading to its applicability in various medicinal and cosmetic fields.

### CROSS-REFERENCE TO RELATED APPLICATION

This application claims priority from and the benefit of Korean Patent Application No. 10-2014-0142624, filed on Oct. 21, 2014, which is herein incorporated by reference for all purposes as if fully set forth herein.

### BACKGROUND

1. Field

The present invention relates to a composition for stabilizing extracellular superoxide dismutase (EC-SOD) protein and a method for stabilizing EC-SOD protein using the composition. More particularly, the present invention relates to a composition for stabilizing EC-SOD protein comprising bovine serum albumin (BSA), human serum albumin (HSA), polyethylene glycol (PEG) or fetal bovine serum (FBS) as an effective ingredient; a method for stabilizing EC-SOD protein using the composition; and a method for preparing a stabilized EC-SOD protein using the composition.

2. Discussion of the Background

Recent investigations of antioxidants on an aging process and efforts of developing related products have drawn attention to human extracellular superoxide dismutase (EC-SOD) (See T. J. Bivalacqua, Am J Physiol Heart Circ Physiol, 284, 1408-1421, 2003; A. G. Est, Free Radic Biol Med, 28, 437-446, 2000).

Superoxide dismutases (SODs) protect cells by removing reactive oxygen species and enabling other antioxidant enzymes to function. SODs include Cu/Zn SOD (SOD 1) containing copper and zinc atoms, Mn SOD (SOD 2) containing a manganese atom, and extracellular SOD (EC-SOD) present on the cell surface or in the extracellular fluid.

Particularly, it has been reported that EC-SODs are effective in treating skin diseases including skin cancer, pigmentation disorder, photo-aging, dermatitis, epidermal hyperplasia, psoriasis, atopy, urticaria and allergy (See Korean Patent No. 100676502), while being also effective in treating and preventing angiogenesis-induced disorders (See Korean Patent No. 101019470). Further, EC-SODs are known to be effective in treating cancers such as colon cancer, lung cancer, liver cancer, stomach cancer, esophageal cancer, pancreatic cancer, gallbladder carcinoma, renal cancer, bladder cancer, prostate cancer, testicular cancer, cervical cancer, endometrial cancer, malignant chorionepithelioma, ovarian cancer, breast cancer, thyroid cancer, brain cancer, head and neck cancer, malignant melanoma, and lymphoma (See Korean Patent Application Publication No. 10-2008-108876).

Thus, for the purpose of being utilized as a composition for treating or preventing said various diseases or disorders, the development of recombinant human EC-SODs have been actively underway by using expression systems of E-coli cells (See H. J. He, Protein Expr Purif, 24, 13-17, 2002; X. Q. Zhu, Acta Biochim Biophys Sin, 37, 265-269, 2005), animal cells (See L, Tibell, Proc Natl Acad Sci, 84, 6634-6638, 1987; M. Stromqvist, J Chromatogr, 621, 139-148, 1993) or yeast (See H. L. Chen, J Agric Food Chem, 54, 8041-8047, 2006; N. Mutoh, Curr Genet, 41, 82-88, 2002). However, since E-coli and yeast cells lack proper post-translational processing systems, it has been recognized that culturing of said human EC-SODs in animal cells is the most preferable.

By the way, recombinant human EC-SODs, which are known to possess an excellent anti-oxidant activity, have drawbacks in their stability and activity. Therefore, when EC-SODs are combined with mineral atoms such as copper (Cu) and zinc (Zn) so as to improve their stability, they reportedly exert their activities stably (See M. C. Carroll, J Biol Chem, v. 281, 28648-28656, 2006; I. M. Ahl, Protein Expr Purif, 37, 311-319, 2004; C. S. Hwang, Microbiology, 148, 3705-3713, 2002; H. Walti, Biol Neonate, 82, 96-102, 2002).

However, regardless of the recent efforts to improve the stability and activity of EC-SODs, there still exists the issue of instability of EC-SODs, leading to an obstacle to the development of EC-SODs in the form of therapeutic or cosmetic compositions. Hence, there has been a necessity for studies to resolve the instability issue of EC-SODs.

### SUMMARY

The present inventors have found that human recombinant EC-SODs can be stabilized by using bovine serum albumin (BSA), human serum albumin (HSA), polyethylene glycol (PEG) or fetal bovine serum (FBS). Hence, the present inventors have completed the present invention by providing a composition and a method for stabilizing extracelluar superoxide dismutase (EC-SOD) protein.

Therefore, one object of the present invention is to provide a composition for stabilizing extracellular superoxide dismutase (EC-SOD) protein.

Another object of the present invention is to provide a method for stabilizing EC-SOD protein.

Further another object of the present invention is to provide a method for preparing a stabilized EC-SOD protein.

The present invention is directed to a composition for stabilizing EC-SOD protein and a method for stabilizing EC-SOD protein using said composition. More specifically, the present invention is directed to a composition for stabilizing EC-SOD protein comprising bovine serum albumin (BSA), human serum albumin (HSA), polyethylene glycol (PEG) or fetal bovine serum (FBS) as an effective ingredient; a method for stabilizing EC-SOD protein using said composition; a method for preparing a stabilized EC-SOD protein using said composition.

The present invention provides a composition for stabilizing EC-SOD protein and a method for stabilizing EC-SOD using said composition in order to use EC-SOD protein for clinical or non-clinical purposes. Further, the present invention attributes to the obtainment of a stabilized EC-SOD protein, leading to its applicability in various medicinal and cosmetic fields.

### BRIEF DESCRIPTION OF THE DRAWINGS

FIG. 1A is a graph showing a fraction obtained by loading a culture solution containing rhSOD3 protein onto nickel column (the arrow indicates purely isolated rhSOD3 protein). FIG. 1B is a picture showing the analysis with reducing and non-reducing SDS-PAGE on rhSOD3- and recombinant 209E-eluted fractions. FIG. 1C shows a data indicating the enzymatic activity (See the graph at the bottom of the figure) and the amount (See the top of the figure) of rhSOD3 protein in which purified rhSOD3 was treated with Cu/Zn or EDTA, denatured, and followed by refolding.

FIG. 2A describes a graph (See the bottom of the figure) showing the measurement of fluorescence signal of 293T-rhSOD3-EGFP after cells were cultured in media containing 0, 10 and 100 μM of Cu/Zn ions, respectively, and an electrophoresis picture (See the top of the figure) measuring the amount of rhSOD3 in culture media. FIG. 2B is a picture showing the Western blot analysis of measuring the amount of intracellular and extracellular rhSOD3 in 293T-rhSOD3 cells.

FIG. 3A is a graph showing the monitoring result of the enzymatic activity of rhSOD3 protein stored in PBS at 4° C., room temperature and 37° C., respectively, for 13 days. FIG. 3B is a graph showing the monitoring result of the enzymatic activity of rhSOD3 protein under the condition of PBS, 0.1% BSA and 10% FBS, respectively, at 37° C. FIG. 3C is a picture showing the Western blot analysis of measuring the amount of rhSOD3 expressed at 4° C., room temperature and 37° C., respectively, after 13 days of cell culture. FIG. 3D shows a graph showing the measurement of the activity of rhSOD3 protein (See the top of the figure) and the Western blot analysis picture showing the measurement of the amount of rhSOD3 protein (See the bottom of the figure) when rhSOD3 protein was kept in PBS buffer (the control group), PBS buffer containing 0.1% BSA, and PBS buffer containing 0.1% PEG, respectively.

FIG. 4 shows graphs showing the monitoring result of the catalytic activity of rhSOD3 (left) and rh209E (right) proteins after repeated freezing/thawing cycles were performed on rhSOD3 and rh209E proteins which were stored in PBS, 0.1% BSA, 1% BSA, 10% glycerol and protein stabilizing cocktail, respectively.

FIG. 5 is a graph showing the monitoring result of the catalytic activity of rhSOD3 protein after repeated freezing/thawing cycles were performed on rhSOD3 protein which was stored in PBS, 0.1% BSA, and 1% HSA, respectively.

FIG. 6 is a graph showing the monitoring result of the catalytic activity of rh209E protein after repeated freezing/thawing cycles were performed on rh209E protein which was stored in PBS, 0.1% BSA, and 1% HSA, respectively.

### DETAILED DESCRIPTION OF THE ILLUSTRATED EMBODIMENTS

Hereinafter, the present invention will be described in detail as below. It is to be understood, however, that these following examples are for illustrative purpose only and are not construed to limit the scope of the present invention.

In order to achieve the above mentioned object, the present invention provides a composition for stabilizing EC-SOD protein comprising at least one selected from the group consisting of bovine serum albumin (BSA), human serum albumin (HSA), polyethylene glycol (PEG) and fetal bovine serum (FBS) as an effective ingredient.

In order to achieve the above mentioned another object, the present invention provides a method for stabilizing EC-SOD protein, the method comprising the step of adding EC-SOD protein into the inventive stabilizing composition.

In order to achieve the above mentioned further object, the present invention provides a method for preparing a stabilized EC-SOD protein, the method comprising the steps of:

(a) transfecting host cells with a plasmid constructed to include an EC-SOD gene, followed by culturing the transfected cells in a culture media;

(b) purifying an extracellular superoxide dismutase (EC-SOD) protein from the culture media cultured in the step (a); and

(c) contacting the purified EC-SOD protein with at least one selected from the group consisting of bovine serum albumin (BSA), human serum albumin (HSA), polyethylene glycol (PEG) and fetal bovine serum (FBS).

The present invention is explained in detail as follows.

Unless otherwise defined, technological and scientific terms as used herein have the same meanings as those understood by one having an ordinary skill in the art, and correspond to the descriptions of the following references (See Singleton et al. (1994) Dictionary of Microbiology and Molecular Biology, 2^{nd} Ed., J. Wiley & Sons, New York, N.Y.; and Janeway, C., Travers, P., Walport, M., Shlomchik (2001) Immunobiology, 5^{th} Ed., Garland Publishing, New York).

The present invention provides a composition for stabilizing superoxide dismutase (EC-SOD) protein comprising at least one selected from the group consisting of bovine serum albumin (BSA), human serum albumin (HSA), polyethylene glycol (PEG) and fetal bovine serum (FBS) as an effective ingredient.

BSA (bovine serum albumin) as used herein is a serum albumin protein derived from cows which is present abundantly in the blood plasma of cows. In one aspect of the present invention, 0.1% or 1% BSA is used in mixture with PBS buffer.

HSA (human serum albumin) as used herein is a serum albumin protein derived from human beings which is present abundantly in the human blood plasma.

PEG (polyethylene glycol) as used herein is a compound which is defined as the chemical formula H—(O—CH₂—CH₂)ₙ—OH and widely used in manufacturing medicines. While PEG is utilized as a polymer to induce cell fusion, 0.1% PEG is used in mixture with PBS buffer in one aspect of the present invention.

FBS (fetal bovine serum) as used herein is the blood fraction of a bovine fetus in which red blood cells are removed. In one aspect of the present invention, 10% FBS is used in mixture with PBS buffer.

Among three kinds of superoxide dismutase (SOD), the extracellular superoxide dismutase proteins are one excreted extracelluarly and abbreviated as EC-SOD or SOD3.

The stabilizing composition as defined herein may comprise water (for instance, filtered or sterilized water) or buffer solution such as acetate buffer solution, succinate buffer solution, phosphate buffer solution and citrate buffer solution.

The proteins as used herein include chemically synthesized proteins, naturally synthesized proteins encoded by genes of cultured cells, and recombinant proteins excreted from cells. Recombinant proteins means proteins encoded by transgenes introduced into cells via molecular biological techniques. The proteins may be modified post-translationally through chemical or enzymatic methods. Preferably, EC-SOD proteins according to the present invention are recombinant proteins.

Regarding protein stabilization, it may be considered that a protein is stabilized even when the activity of a protein is lower in comparison with its naturally occurring state or control group, or when its lowered activity further decreased during its storage. Moreover, it may be considered that a protein is stabilized when decomposition or denaturalization of a protein is inhibited. As a specific example of protein stabilization, whether the activity of EC-SOD is increased or not may be confirmed by evaluating the activities of control and test groups under the same condition.

The activity of a protein as used herein includes, but not limited thereto, coupling, neutralization, cell damaging, agonistic, antagonistic, enzymatic, catalytic activities. Preferably, it may be an activity inducing quantitative and/or qualitative change or effect in a subject's body, systems, cells, proteins, DNAs, RNAs and so on. More preferably, it may be a catalytic activity.

The composition for stabilizing extracellular superoxide dismutase (EC-SOD) protein according to the present invention is characterized by stabilizing its catalytic activity. The catalytic activity of EC-SOD protein as used herein means the activity or function of EC-SOD protein to remove superoxide (O₂⁻) and thus prevent the occurrence of mutation. The catalytic activity of EC-SOD protein according to the present invention may be described as the following reaction formula in which metal ions and EC-SOD are coupled to remove superoxides and produce oxygens, or superoxides are reacted with hydrogens to produce hydrogen peroxides.

[Formula 1]

M⁽ⁿ⁺¹⁾⁺—SOD+O₂⁻→Mn+—SOD+O₂ 1)

Mⁿ⁺—SOD+O₂+2H⁺→M⁽ⁿ⁺¹⁾+—SOD+H₂O₂ 2)

* M=Cu (n=1); Mn (n=2); Fe (n=2); Ni (n=2)

In one embodiment of the present invention, the activity of scavenging superoxide may be measured in order to determine the catalytic activity of rhSOD3.

The EC-SOD protein according to the present invention may be characterized in that it is a recombinant protein.

The recombinant proteins as used herein include not only peptides, polypeptides, proteins, oligoproteins and/or fusion proteins, but also any proteins expressed from amino acid-encoding recombinant genetic materials or biologically active portions thereof (e.g. a portion possessing the biological activity of a protein in its entirety). Recombinant protein products may include therapeutic, preventative or diagnostic products.

The recombinant proteins as described above may be characterized in that they are obtained from cells transfected with plasmids constructed to include EC-SOD gene.

The plasmid as described above means a nucleic acid molecule that can carry another nucleic acids to which it is connected. That is, the plasmid means a circular, double-stranded DNA molecule inside which additional DNA segments may be ligated. In one aspect of the present invention, pcDNA3.1 vector was employed to obtain cells producing recombinant proteins. Since the plasmid is the most commonly used vector, plasmids and vectors are used interchangeably in the detailed description of the present application.

The EC-SOD of the present invention is characterized in that it is human EC-SOD gene of SEQ ID NO: 1.

The present invention provides a method for stabilizing EC-SOD protein, the method comprising the step of adding EC-SOD protein into the inventive stabilizing composition.

The EC-SOD protein according to the present invention is characterized by being recombinant protein.

The recombinant proteins as used herein include not only peptides, polypeptides, proteins, oligoproteins and/or fusion proteins, but also any proteins expressed from amino acid-encoding recombinant genetic materials or biological active portions thereof (e.g. a portion possessing the biological activity of a protein in its entirety). Recombinant protein products may include therapeutic, preventative or diagnostic products.

The EC-SOD protein according to the present invention is characterized in that it is prepared by a method comprising the steps of:

(a) transfecting host cells with a plasmid constructed to include an EC-SOD gene, followed by culturing the transfected cells in culture media; and

(b) purifying an extracellular superoxide dismutase (EC-SOD) protein from the culture media cultured in the step (a).

The term “transfecting” as used herein describes introducing a genetic information into an organism, particularly eukaryotic cells. “Transfecting” comprises introducing a genetic information, especially DNA, by using any available known methods in the art including, but not limited thereto, bombardment with DNA-enriched particles, protoplast transfection, DNA microinjection, electroporation, fusion or transfection of competent cells, chemical—or agrobacteria-mediated transfection. The genetic information as described herein refers to, but not limited thereto, a genetic domain, a gene or a plurality of genes. The genetic information may be introduced into a target cell with the aid of a vector or free nucleic acid (for example, DNA and RNA) or any other means, resulting in that it may be incorporated into a host genome through recombination or exist in its free form.

The cell as used herein is characterized by being a mammalian cell. Regarding the cell as used herein, prokaryotic cells such as bacterial cells or yeasts do not possess post-translational processing systems, causing a difficulty in producing active EC-SOD proteins. Therefore, the cell as used herein is preferably a mammal cell including livestock and human cell.

Preferably, the step of culturing the transformed cells in culture media in order to prepare EC-SOD as described in the present invention is carried out without further addition of CU²⁺ or Zn²⁺ alone or in combination thereof. As found by the present inventors, Cu²⁺ or Zn²⁺ ions inhibit the extracellular secretion of EC-SOD from the transfected cells, leading to a decrease in the overall yield of EC-SOD.

In the above cell culture, the concentration of Cu²⁺ ions may be preferably 0.01 to 10 mM, more preferably 0.03 to 1 mM, still more preferably 0.04 to 0.1 mM, while the concentration of Zn²⁺ ions may be preferably 0.01 to 10 mM, more preferably 0.03 to 1 mM, still more preferably 0.04 to 0.1 mM.

The present invention provides a method for preparing a stabilized EC-SOD protein, the method comprising the steps of:

(a) transfecting host cells with a plasmid constructed to include an EC-SOD gene, followed by culturing the transfected cells in culture media;

(b) purifying an extracellular superoxide dismutase (EC-SOD) protein from the culture media cultured in the step (a); and

(c) contacting the purified EC-SOD protein with at least one selected from the group consisting of bovine serum albumin (BSA), human serum albumin (HSA), polyethylene glycol (PEG) and fetal bovine serum (FBS).

The purification step (b) may be carried out by utilizing any protein purification techniques known in the art including chromatography (such as affinity chromatography, ion exchange chromatography, liquid chromatography, gel filter chromatography), dialysis and desalting. One example according to the present invention utilized a chromatography purification technique employing HiTrap chelating HP columns.

Preferably, the EC-SOD according to the present invention may be purified by using a buffer containing at least one selected from the group consisting of bovine serum albumin (BSA), human serum albumin (HSA), polyethylene glycol (PEG) and fetal bovine serum (FBS). One example according to the present invention utilized the addition of BSA, HSA, PEG and FBS at the end of dialysis to further stabilize the EC-SOD.

the term “contacting” as used herein means mixing EC-SOD protein in a solution comprising the stabilizing composition according to the present invention. The solution may further contain another ingredient such as a buffer. The composition according to the present invention may be added into media soaked with the protein via a delivery device (such as a pipet-based or syringe-based device).

The stabilized EC-SOD according to the present invention maintain its stability under freezing or thawing condition.

The freezing condition as used herein includes quick freezing and slow freezing, while the thawing condition also includes quick thawing and slow thawing. For example, the protein according to the present invention may be kept stabilized at about 2° C. to 8° C. in some instances. In a specific embodiment, the protein may be kept at about −80° C. In case where the protein is frozen, the inventive method may include the step of quickly thawing the protein. If the protein is required to be frozen (for instance, for the purpose of its storage), the protein may be frozen through quick freezing method. In some aspects, slow freezing method may be employed. The composition according to the present invention is characterized by maintaining the activity of the protein under such a changing temperature.

The method for preparing the stabilized EC-SOD protein according to the present invention is characterized by maintaining the activity of the protein under repeated freezing and thawing conditions.

As described in one example of the present application, rhSOD3 or rh209E proteins in combination with media containing the inventive composition were frozen using liquid nitrogen, kept at −80° C. and then thawed. Even after this process was repeated four times, it was found that said proteins kept their stable activity almost intact. In contrast, it was shown that the activity of the proteins without the inventive composition decreased more than 50% even after a single freezing/thawing cycle.

Preferably, the freezing condition as described herein may be freezing with liquid nitrogen, followed by being kept at −80° C. The thawing condition as described herein may be room temperature (i.e. 20° C. to 40° C., preferably 37° C.).

The present invention is characterized in that the stabilized EC-SOD protein according to the present invention maintains its activity for thirteen (13) days or longer.

In accordance with one example of the inventive method for preparing the stabilized EC-SOD protein, rhSOD3 protein keeps its stable activity for thirteen days or longer at 37° C. under the condition of containing the inventive composition.

The stabilized EC-SOD protein according to the present invention is characterized by maintaining its activity at high temperature.

One example of the present application showed a result of monitoring the catalytic activity of rhSOD3 and rh209E proteins under the condition of PBS, 0.1% BSA, 0.1% HSA or 10% FBS at 37° C., respectively. It was found that rhSOD3 and rh209E proteins maintained 80% of their catalytic activity after thirteen(13) days under the condition containing the inventive composition, whereas the control groups showed 40% decrease in their catalytic activity, i.e. 60% of their catalytic activity. By the way, the term “high temperature” as described above may be 35° C. to 45° C., preferably 36° C. to 40° C., more preferably 36.5° C. to 38° C., most preferably 37° C.

### <Experimental Methods>

Cloning and Mammalian Cell Culture

The full length human SOD3 protein corresponds to Met1 to Glu227 as represented in SEQ ID NO: 2, while 209E variant protein as represented in SEQ ID NO: 4 has the heparin-binding domain of human SOD3 protein deleted at its C-terminal end. Both proteins contain His6tag or enhanced green fluorescence protein (EGFP) at their C-terminal ends. In order to prepare said proteins, hSOD3 gene as represented in SEQ ID NO: 1 and h209E variant gene as represented in SEQ ID NO: 3 were inserted into pcDNA3.1 (Invitrogen) using HindIII and EcoRI or HindIII and XbaI, respectively. Plasmids encoding hSOD3 and 209E variant were respectively transfected into 293T-EBNA cells with Attractene (Qiagen) based on the manufacturer's instructions, respectively. 24 hours after transfection, the culture media were replaced with serum-free Dulbecco's Modified Eagle Medium (DMEM). 293T-EBNA cells stably expressing rhSOD3-EGFP were selected using G418 (Invitrogen) and enriched by fluorescence-activated cell sorting (FACS). 293T-EBNA and Raw264.7 cells were maintained in DMEM media containing 10% FBS.

Protein Expression and Purification

Five days after transfection, the culture media containing rhSOD3 (recombinant human superoxide dismutase 3) were collected, and then loaded onto HiTrap Chelating HP column (GE Healthcare) to be filtered. After loading, the column was washed with more than 50 column volumes of a washing buffer (50 mM NaPO₄, 500 mM NaCl and 30 mM imidazole). Subsequently, rhSOD3 and 209E proteins were eluted by elution buffer containing 500 mM imidazole, followed by dialysis in PBS or said washing buffer (50 mM NaPO₄, 500 mM NaCl and 30 mM imidazole). The concentration of purified rhSOD3 was determined based on a BSA standard curve with protein assay dye (Bio-Rad).

Activity Assay for SOD

To measure the enzymatic activity of rhSOD3, the rate of superoxide radical formation was quantified spectrophotometically. A 20 μl sample was mixed with 200L of 200M xanthine (Sigma) and 50M WST-1 (Dojindo) in PBS. After adding 0.0005 unit XOD (Sigma), formazan dye signal was measured using a colorimetric method. The generation of formazan dye signal was determined kinetically, and absolute SOD activity was determined from the dilution factor exhibiting 75% inhibition (IC₅₀) on the inhibition curve.

Anti-Inflammatory Effects of rhSOD3

At 70% confluence, Raw264.7 cells were starved on serum-free DMEM media for 6 hours and then treated with 1 μg/μl of lipopolysaccharide (LPS). The amount of rhSOD3 added to cells was determined in accordance with a ratio of holo-enzyme to apo-enzyme, but maintained its constant amount corresponding to 100 units of 100% holo-enzyme/μl. A SDS sample buffer containing protease inhibitors were directly added to cells which were then incubated for 24 hours. iNOS, GAPDH, and hSOD3 were analyzed by Western blot assay with anti-NOS2 (Santa Cruz Biotechnology), anti-GAPDH (Santa Cruz Biotechnology), and anti-hSOD3 (AbCam) antibodies.

Monitoring Activity of rhSOD3

Purified rhSOD3 was incubated in different culture conditions using 0.1% BSA, 0.1% HSA or 10% FBS at 4° C., room temperature (RT) or 37° C., respectively. Activity of 10 μl of purified rhSOD3 corresponding to 5.6 U initial activity was monitored for 13 days. To assess the effect of freezing/thawing on the activity of rhSOD3, purified rhSOD3 supplemented with 1% BSA, 0.1% BSA, 0.1% HSA, 10% glycerol or protein stabilizing cocktail (Thermo Scientific) were quickly frozen with liquid nitrogen, respectively. After thawing, the activity of 2 μl of rhSOD3 corresponding to 1 U initial activity was determined.

Supplementation of Copper and Zinc Ions and Refolding

In order to determine the effect of Cu/Zn on the expression of rhSOD3, 0M, 10M and 100M CuSO₄/ZnCl₂ mixture were respectively added to the culture media for 293T cells stably expressing rhSOD3. 24 hours later, the amount of expressed and secreted rhSOD3 in the culture media was determined by Western blot assay with anti-SOD3 antibody. Further, the amount of rhSOD3-EGFP was measured with GFP fluorescence. To assess the effect of Cu/Zn on purified rhSOD3, purified rhSOD3 was directly mixed with 50M CuSO₄/ZnCl₂, or dialyzed into PBS containing 10M CuSO₄/ZnCl₂. Subsequently, free Cu/Zn were removed. For refolding, rhSOD3 was denatured by 6M Guanidine HCl and then was refolded by dialysis into PBS containing 10M CuSO₄/ZnCl₂ or 10 mM EDTA.

### <Experimental Results>

Purification of rhSOD3

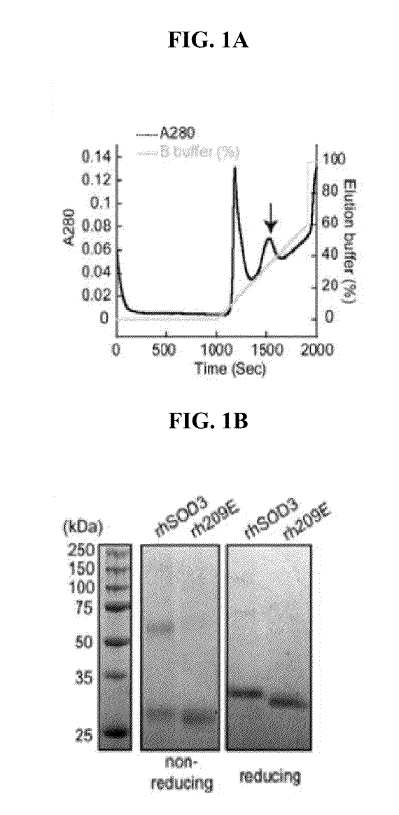

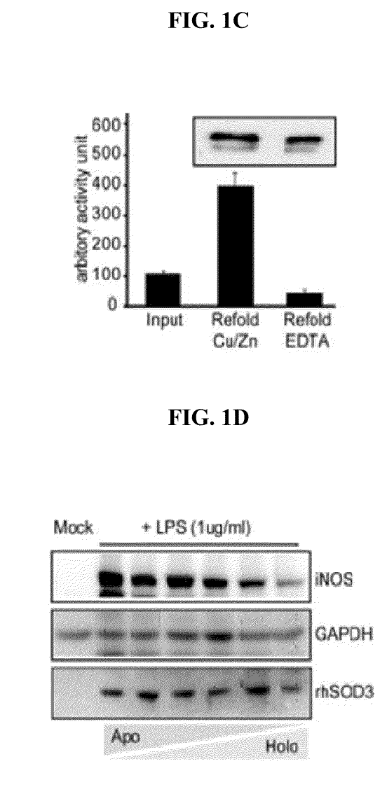

rhSOD3 tagged with C-terminal His₆ was purified directly from culture media on a nickel column (See FIG. 1A). Purified rhSOD3 showed around 27 kDa of monomer size and a half of rhSOD3 formed a dimer via intermolecular disulfide bond, whereas recombinant 209E did not show dimeric band in non-reducing SDS-PAGE (See FIG. 1B). Based on these results, it is concluded that the heparin-binding domain is needed for SOD3 to form a dimer.

Catalytic Activity of rhSOD3

SOD3 is a metalloenzyme that uses copper and zinc ions as cofactors for catalysis. If the basal amount of metal ions in a culture media is insufficient to accommodate over-expressed rhSOD3, metal-free rhSOD3 may be secreted to the culture media. Cells have chaperones CCS1 and ATOX1 to assist the incorporation of copper into SOD1 and SOD3, respectively. However, SOD3 has been shown to combine with metal cofactors under in vitro condition without chaperone proteins. Therefore, the inventors expected that copper/zinc ions could enhance catalytic activity of purified rhSOD3. Immediately after elution from nickel column, rhSOD3 was dialyzed into either PBS or PBS containing 50 M Cu/Zn ions, followed by additional dialysis of unbound metal ions into PBS. It was found that rhSOD3 dialyzed into a buffer containing Cu/Zn ions was much more active than one dialyzed into a plain PBS buffer (Data not shown). This result suggests that over-expressed rhSOD3 is either secreted from cells in a metal-free form or loses metal ions after its secretion, resulting as an apo-enzyme.

In order to estimate approximate percentage of apo-enzyme in the purified sample, purified rhSOD3 was denatured, and refolded in the presence of a metal chelator, EDTA to maximize the percentage of apo-enzyme, or in the presence of Cu/Zn ions to maximize the percentage of holo-enzyme. Refolded rhSOD3 in the presence of Cu/Zn ions showed almost four(4) times higher activity in comparison with the initial activity of purified rhSOD3 (See FIG. 1C). In contrast, refolded rhSOD3 in the presence of EDTA showed less activity in comparison with the initial activity of purified rhSOD3. rhSOD3 did not show any visible aggregation during refolding in any condition (Data now shown). Further, the amount of protein was similar in both conditions (See enclosed figure of FIG. 1C). These results suggest that around 75% of purified rhSOD3 is apo-enzyme or unfolded enzyme which is able to recover its activity by post-translational metal incorporation and refolding.

Analysis of relationship between anti-inflammatory effect and enzymatic activity of rhSOD3

Although rhSOD3 has been reported to not possess its enzymatic activity against asthma, it is considered that its enzymatic activity would lead to its anti-inflammatory effect on ROS (reactive oxygen species)-induced inflammation. LPS(Lipopolysaccharide)-activated cells showed significant induction of iNOS, one of inflammatory markers Inhibitory effect of rhSOD3 on iNOS induction decreased as the percentage of its holo-enzyme was lowered (See FIG. 1D). This correlation between the enzymatic activity of rhSOD3 and its efficacy emphasizes the importance of the enzymatic activity of rhSOD3 for its future application.

Effect of Metal Ions on rhSOD3 Expression

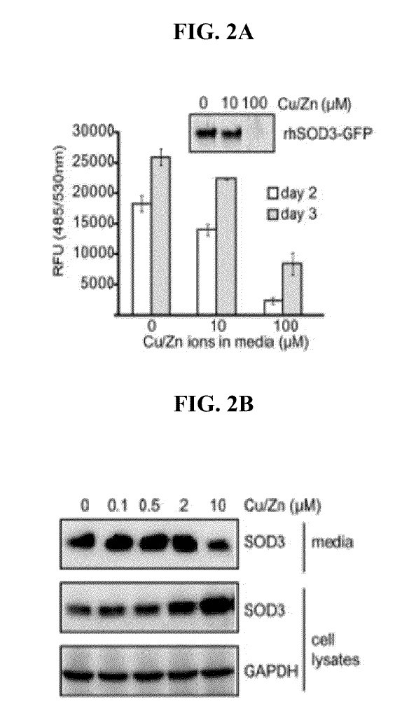

It was described above that over-expressed rhSOD3 in a culture media with insufficient metal ions could be secreted without metal ions. Thus, the inventors investigated whether the supplement of Cu/Zn ions might increase the expression level of active rhSOD3. Unexpectedly, the supplement of Cu/Zn ions significantly reduced the secretion of rhSOD3. It was found that extracellular fluorescent signals of rhSOD3-GFP decreased at a high concentration of Cu/Zn ions in culture media, and the Western blot analysis confirmed this result (See FIGS. 2A and 2B). Instead, rhSOD3 seemed to accumulate inside cells under high concentration of metal ions (See FIG. 2B), suggesting that Cu/Zn ions might inhibit secretion or induce internalization of rhSOD3. Thus, the inventors concluded that the supplementation of Cu/Zn ions to culture media might not enhance the expression of active rhSOD3.

Stabilization of Active rhSOD3

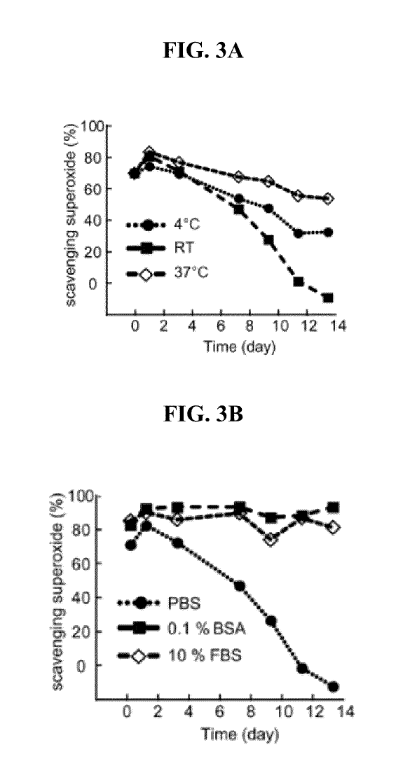

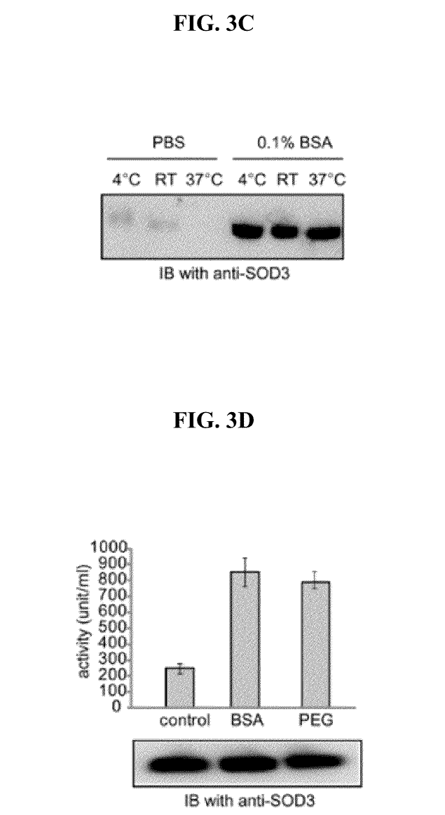

The inventors noted that rhSOD3 was very unstable after purification procedures. It was found that purified rhSOD3 lost almost half of its initial activity in PBS buffer at 37° C. within seven(7) days (See FIG. 3A). The loss of its activity was also detected even at a lower temperature. Thus, it is necessary to optimize formulations and storage conditions for stabilizing active rhSOD3.

First, purified rhSOD3 was supplemented with 0.1% BSA or 10% FBS and maintained in PBS buffer. The inventors confirmed that the supplementation of either 0.1% BSA or 10% FBS stabilized the activity of rhSOD3. It was found that rhSOD3 maintained its catalytic activity for up to 13 days at 37° C. (See FIG. 3B). Purified rhSOD3 was completely decomposed after 13 days in PBS buffer, whereas rhSOD3 remained intact in PBS buffer supplemented with 0.1% BSA (See FIG. 3C).

Second, the inventors examined whether rhSOD3 lost its activity over the course of purification procedures at a low temperature for 2 days. Right after elution from affinity column, purified rhSOD3 was mixed with PBS and 0.1% BSA, followed by dialysis into PBS buffer or PBS buffer containing 0.1% PEG and another protein stabilizer. Interestingly, rhSOD3 prepared through dialysis with 0.1% BSA or 0.1% PEG showed much higher activity than one prepared through dialysis with PBS only (See FIG. 3D). The amount of protein obtained was not affected by the type of dialysis (See FIG. 3D), suggesting that BSA or PEG might prevent the release of metal ions from purified active rhSOD3.

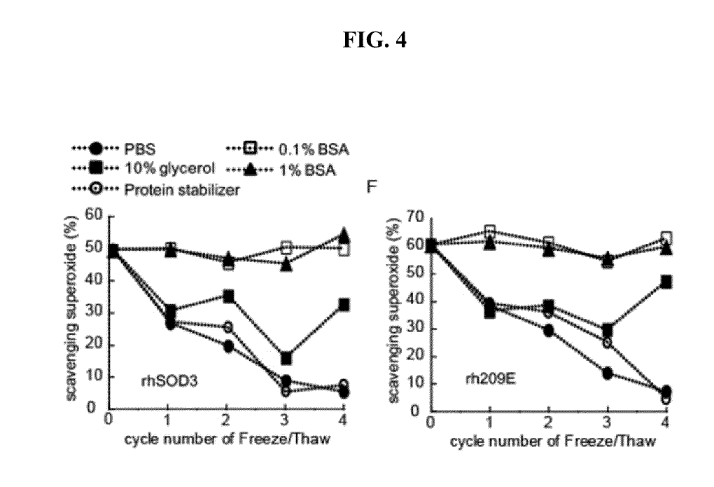

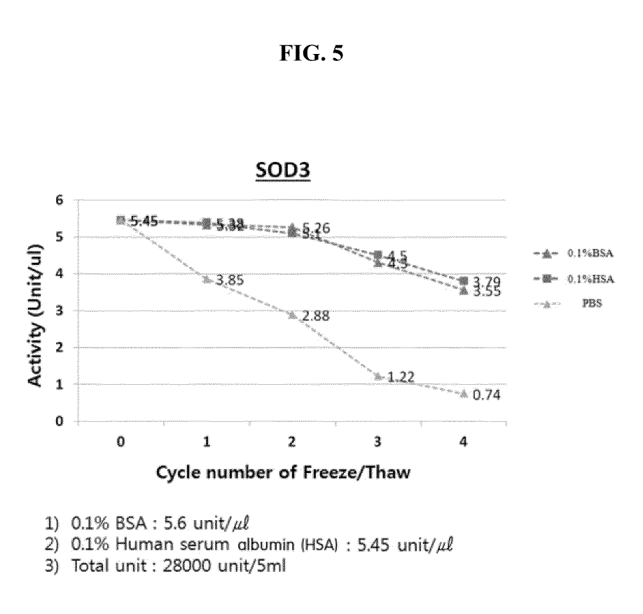

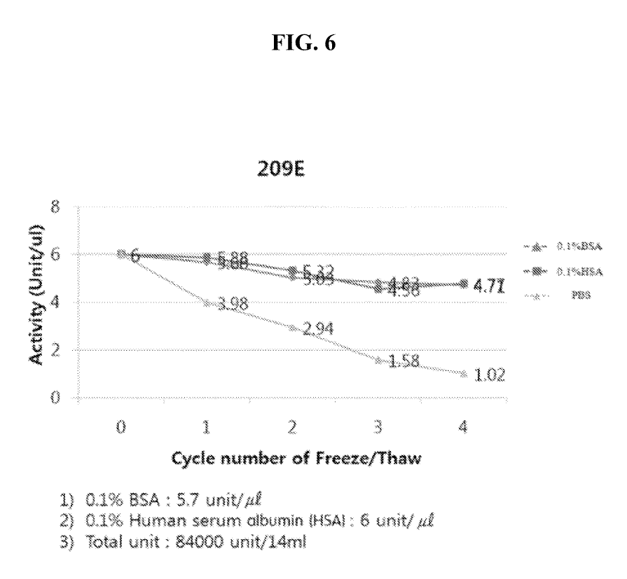

Lastly, the effect of freezing/thawing cycles on rhSOD3 and 209E proteins was then investigated. In general, repeated freezing/thawing has been known to destabilize proteins. Right after purification procedures, purified rhSOD3 and 209E proteins were frozen with liquid nitrogen and then stored at −80° C. under different buffer conditions, respectively. Immediately after thawing, the catalytic activity of each protein was determined. rhSOD3 and 209E proteins stored in PBS lost half of their activities after a single cycle of freezing/thawing. On the contrary, BSA (1% or 0.1%) prevented the activity loss of rhSOD3 and 209E proteins due to freezing/thawing cycles (See FIG. 4). 10% glycerol partially stabilized the activity of rhSOD3, while 0.1% HSA also stabilized the activity of rhSOD3 (See FIGS. 5 & 6). However, it was found that the commercially available protein stabilizing cocktail failed to stabilize the activity of rhSOD3.

1. A composition for stabilizing extracellular superoxide dismutase (EC-SOD) protein comprising at least one selected from the group consisting of bovine serum albumin (BSA), human serum albumin (HSA), polyethylene glycol (PEG) and fetal bovine serum (FBS) as an effective ingredient.

2. The composition of claim 1, wherein the composition stabilizes the catalytic activity of EC-SOD protein.

3. The composition of claim 1, wherein the EC-SOD protein is a recombinant protein.

4. The composition of claim 3, wherein the recombinant protein is obtained from cells transfected with a plasmid constructed to include EC-SOD gene.

5. The composition of claim 1, wherein the EC-SOD protein is encoded by human EC-SOD gene represented by a sequence of SEQ ID NO: 1.

6. A method for stabilizing extracellular superoxide dismutase (EC-SOD) protein, the method comprising adding the EC-SOD protein into the composition according to claim 1.

7. The method of claim 6, wherein the EC-SOD protein is a recombinant protein.

8. The method of claim 7, further comprising preparing the EC-SOD protein,
wherein preparing of the EC-SOD protein comprises:
transfecting host cells with a plasmid constructed to include an EC-SOD gene, followed by culturing the transfected cells in culture media; and
purifying the EC-SOD protein from the culture media cultured in the culturing.

9. The method of claim 7, wherein the cell is mammalian cell.

10. A method for preparing a stabilized extracellular superoxide dismutase (EC-SOD) protein, the method comprising:
transfecting host cells with a plasmid constructed to include an EC-SOD gene, followed by culturing the transfected cells in culture media;
purifying the EC-SOD protein from the culture media cultured in the culturing; and
contacting the purified EC-SOD protein with at least one selected from the group consisting of bovine serum albumin (BSA), human serum albumin (HSA), polyethylene glycol (PEG) and fetal bovine serum (FBS).

11. The method of claim 10, wherein the stabilized EC-SOD protein maintains its activity under the condition of freezing or thawing.

12. The method of claim 11, wherein the stabilized EC-SOD protein maintains its activity under the condition of repeated freezing or thawing.

13. The method of claim 10, wherein the stabilized EC-SOD protein maintains its activity for thirteen (13) days or longer.

14. The method of claim 10, wherein the stabilized EC-SOD protein maintains its activity at a high temperature.

15. The composition of claim 2, wherein the EC-SOD protein is encoded by human EC-SOD gene represented by a sequence of SEQ ID NO: 1.

16. The composition of claim 3, wherein the EC-SOD protein is encoded by human EC-SOD gene represented by a sequence of SEQ ID NO: 1.

17. The composition of claim 4, wherein the EC-SOD protein is encoded by human EC-SOD gene represented by a sequence of SEQ ID NO: 1.
